(19) United States
(12) Patent Application Publication
(10) Pub. No.: US 2010/0081886 A1
(43) Pub. Date: Apr. 1, 2010

(54) SURGICAL MANIPULATOR SYSTEM

(75) Inventor: Takahiro KOMURO, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/566,203

(22) Filed: Sep. 24, 2009

(30) Foreign Application Priority Data
Sep. 30, 2008 (JP) 2008-254639

(51) Int. Cl. A61B 1/32; A61B 19/00

(52) U.S. Cl. 600/229; 606/130

(57) ABSTRACT

Movement of an instrument to a target treatment site can be performed easily and quickly, so that the burden imposed on an operator can be reduced. A surgical manipulator system including a stabilizer manipulator having a stabilizer, at the tip, holding an operative site of a patient; one or more operating manipulator having an instrument, at the tip, treating the operative site held by the stabilizer manipulator; and a control unit controlling these manipulators, wherein the stabilizer manipulator and the operating manipulator have coordinate systems that are associated with one another; and the position coordinates of a target site to be treated are set to the stabilizer provided on the stabilizer manipulator; and the control unit controls the operating manipulator so as to dispose the instrument in the vicinity of the target site set to the stabilizer when the stabilizer is disposed at the operative site to hold the operative site.

### BACKGROUND OF THE INVENTION

1. Field of the Invention

The present invention relates to a surgical manipulator system.

This application is based on Japanese Patent Application No. 2008-254639, the content of which is incorporated herein by reference.

2. Description of Related Art

In the related art, there is a known surgical manipulator system that is equipped with a plurality of master-slave manipulators that operate in accordance with the input from an operating unit (for example, refer to U.S. Pat. No. 6,398,726).

This surgical manipulator system is equipped with a stabilizer as an end effector of the manipulators, and treatment is carried out using the instrument of another manipulator with viewing an endoscopic image while inhibiting the pulsation of the operative site, in particular, an organ, such as a heart, that involves considerable spontaneous movement, by holding the stabilizer serving as a manipulator against the operative site.

In such a surgical manipulator system, the operator performs the treatment with visually checking the endoscopic image to identify the positional relationship between a site to be treated and the manipulators and considering the direction and distance; therefore, it sometimes takes much time to move the instrument to the treatment site. In particular, in a case where there are a plurality of treatment sites or in a case where it is necessary to move the instrument back and forth multiple times to a single treatment site, a heavy burden is imposed on the operator.

### BRIEF SUMMARY OF THE INVENTION

The present invention provides a surgical manipulator system that can easily and quickly move the instrument to a treatment site of an operative site and that can reduce the burden imposed on the operator.

One aspect of the present invention is a surgical manipulator system including a stabilizer manipulator having a stabilizer, at the tip, holding an operative site of a patient; one or more operating manipulator having an instrument, at the tip, treating the operative site held by the stabilizer manipulator; and a control unit controlling these manipulators, wherein the stabilizer manipulator and the operating manipulator have coordinate systems that are associated with one another; and the position coordinates of a target site to be treated are set to the stabilizer provided on the stabilizer manipulator; and the control unit controls the operating manipulator so as to dispose the instrument in the vicinity of the target site set to the stabilizer when the stabilizer is disposed at the operative site and holds the operative site.

According to the above aspect, when the stabilizer manipulator is operated to hold the operative site with the stabilizer so that the target site set to the stabilizer matches the treatment site of the operative site, the target site set to the stabilizer is associated with the treatment site of the operative site. Since the coordinate systems of the stabilizer manipulator and the operating manipulator are associated with each other, the control unit can control the operating manipulator so as to dispose the instrument in the vicinity of the target site associated with the treatment site of the operative site, thus allowing the instrument to be moved to the treatment site easily and quickly. As a result, even in a case where there are a plurality of treatment sites or in a case where it is necessary to move the instrument back and forth multiple times to a single treatment site, the burden imposed on the operator can be reduced.

In the above aspect, the surgical manipulator system may further include an observation manipulator having a camera, at the tip, for observing the operative site and having a coordinate system that is mutually associated with the stabilizer manipulator; a display unit displaying an image of the operative site acquired by the camera of the observation manipulator; and an indicator display unit displaying an indicator for specifying the treatment site in the image displayed on the display unit.

With this configuration, when the operator operates the stabilizer manipulator in a state in which the operator operates the observation manipulator and acquires an image of the operative site with the camera, the stabilizer moves in the image displayed on the display unit. Since the display unit displays an indicator using the indicator display unit, the operator can move the stabilizer using the indicator in the displayed image so that the target site set to the stabilizer matches the treatment site of the operative site. The operator can easily immobilize the target site to the treatment site of the operative site by holding the stabilizer against the operative site, with the target site aligned with the treatment site specified by the indicator.

In the above aspect, the indicator may be a shaped portion provided on the surface of the stabilizer.

In the above aspect, the shaped portion may be a scale provided on the surface of the stabilizer.

In the above aspect, the shaped portion may be a characteristic shape formed on the main body of the stabilizer.

With this configuration, when the stabilizer is moved, the shaped portion (scale, characteristic portion) drawn on the surface of the stabilizer is also moved at the same time, and thus, the stabilizer manipulator can be operated so that the target site can easily be aligned with the treatment site specified by the shaped portion.

In the above aspect, the indicator display unit may superimpose an image of the indicator set to the stabilizer on the image of the operative site.

Furthermore, the image of the indicator may be a scale.

With this configuration, the image of the indicator (scale) superimposed in the image of the operative site displayed on the display unit moves together with the stabilizer, and thus, the treatment site specified by the indicator can easily be aligned with the target site of the stabilizer.

The present invention offers the advantages of easily and quickly moving the instrument to a treatment site of the operative site and reducing the burden imposed on the operator.

### BRIEF DESCRIPTION OF THE SEVERAL VIEWS OF THE DRAWINGS

FIG. 1 is a perspective view showing a surgical manipulator system according to an embodiment of the present invention;

FIG. 2 is a block diagram showing the surgical manipulator system in FIG. 1;

FIG. 3 is a perspective view showing an observation manipulator provided in the surgical manipulator system in FIG. 1;

FIG. 4 is a perspective view showing a stabilizer manipulator provided in the surgical manipulator system in FIG. 1;

FIG. 5 is a perspective view showing an operating manipulator provided in the surgical manipulator system in FIG. 1;

FIG. 6A is a perspective view showing an example of a stabilizer provided on the stabilizer manipulator in FIG. 4;

FIG. 6B is a back view showing an example of the stabilizer provided on the stabilizer manipulator in FIG. 4;

FIG. 6C is a plan view showing an example of the stabilizer provided on the stabilizer manipulator in FIG. 4;

FIG. 7 is a plan view showing an example of an indicator provided on the stabilizer in FIGS. 6A to 6C;

FIG. 8 is a perspective view showing the state of treatment of an operative site by the surgical manipulator system in FIG. 1;

FIG. 9 is a plan view showing a modification of the indicator in FIG. 7; and

FIG. 10 is a plan view showing another modification of the indicator in FIG. 7.

### DETAILED DESCRIPTION OF THE INVENTION

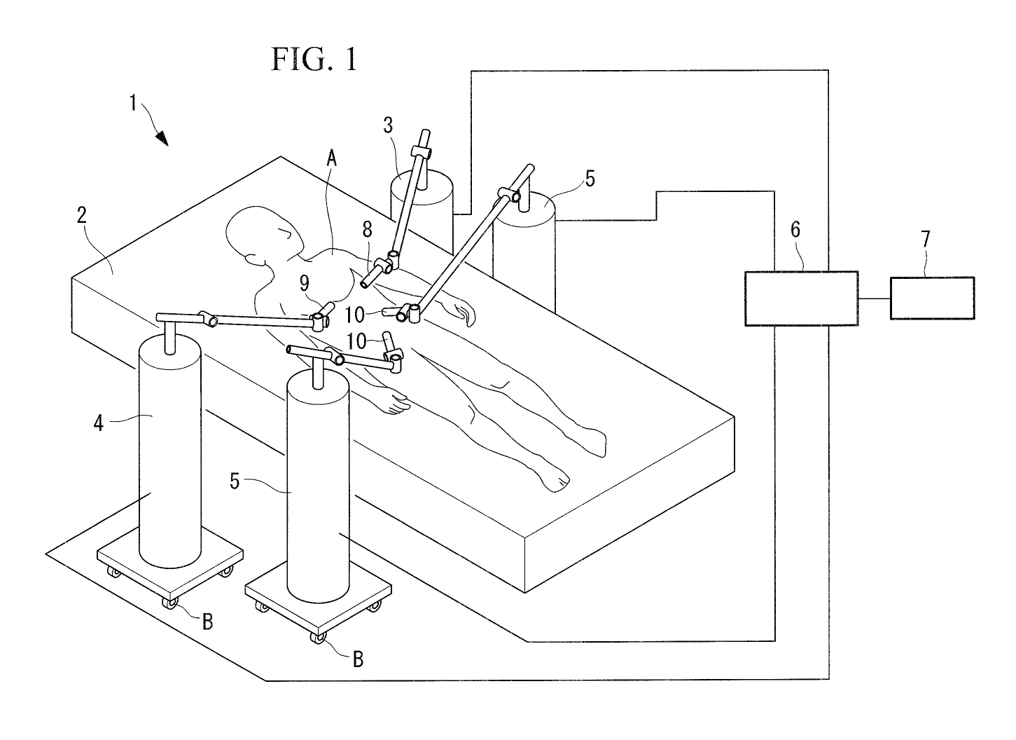

A surgical manipulator system 1 according to an embodiment of the present invention will be described below with reference to FIGS. 1 to 8.

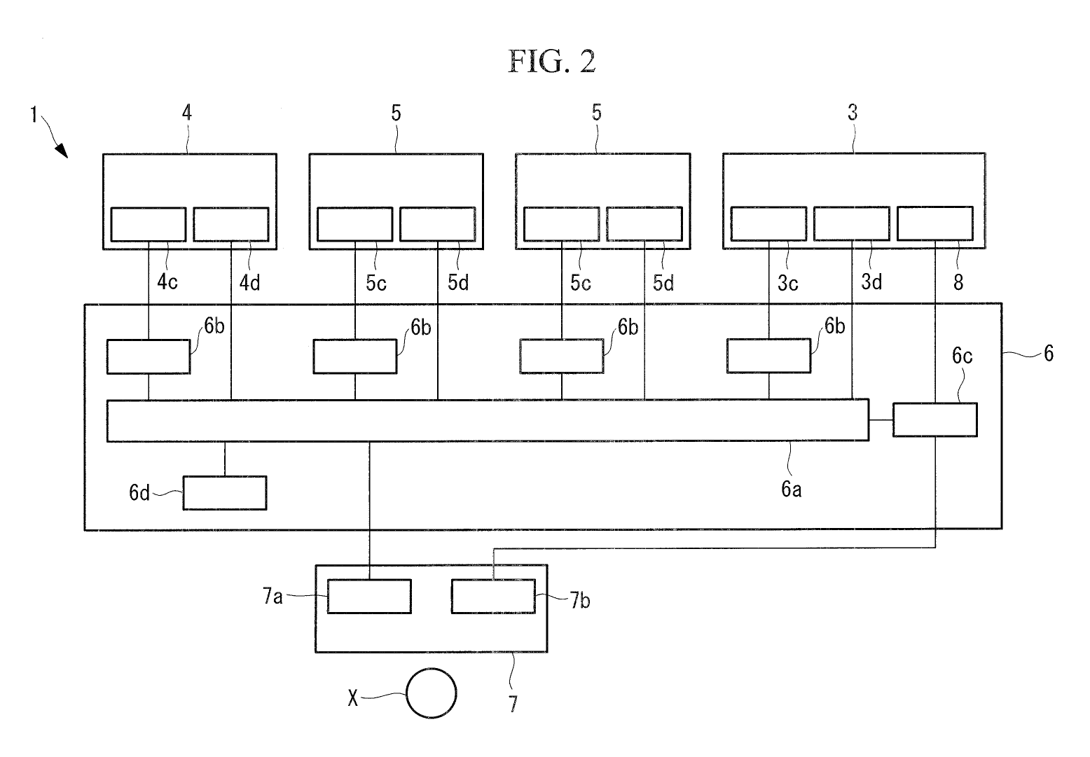

As shown in FIGS. 1 and 2, the surgical manipulator system 1 according to this embodiment is equipped with a plurality of (in the illustrated example, four) manipulators 3, 4, and 5 that are movably disposed, using casters B, around a bed 2 on which a patient A is laid; a control unit 6 connected to the manipulators 3 to 5; and an operating unit 7 that inputs operating signals from the manipulators 3 to 5 to the control unit 6.

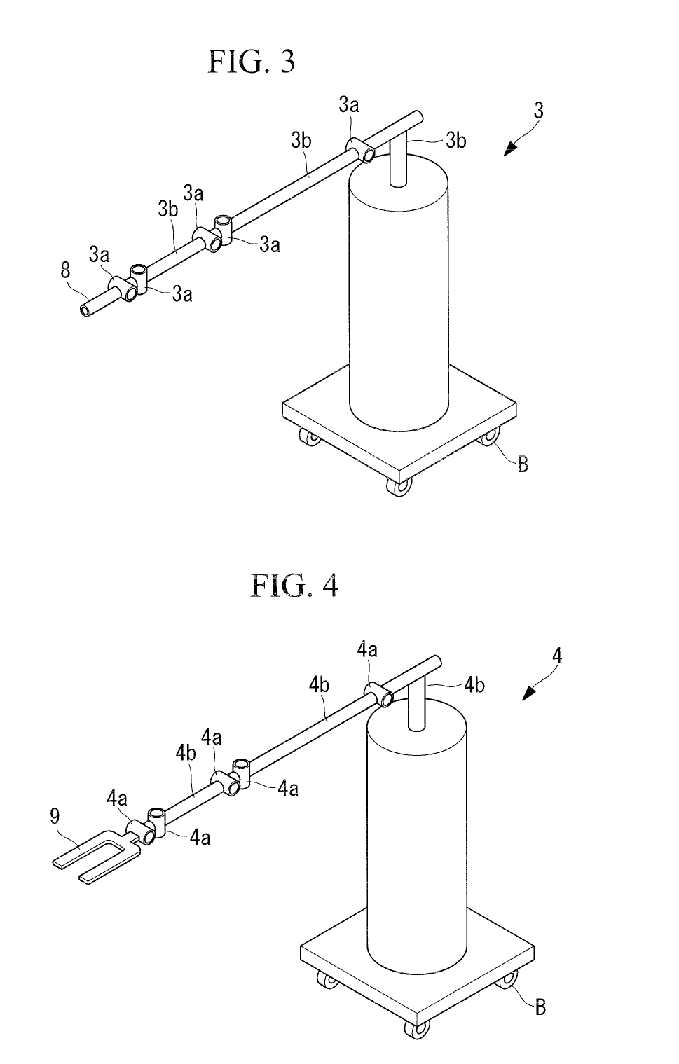

As shown in FIGS. 3 to 5, the manipulators 3 to 5 are of an articulated type (for example, a six-axis articulated type) in which the leading-end position can be oriented in any direction and have a plurality of links 3b to 5b connected together with a plurality of joints 3a to 5a. As shown in FIG. 2, the joints 3a to 5a are equipped with driving units (motors and reduction gears) 3c to 5c and sensors 3d to 5d that detect the rotation angles of the links 3b to 5b. The example shown in FIG. 2 collectively shows the driving units 3c to 5c and the sensors 3d to 5d provided for the plurality of joints 3a to 5a, respectively.

As shown in FIG. 3, of the four manipulators 3 to 5, one manipulator 3 is an observation manipulator (hereinafter also referred to as an observation manipulator 3), equipped with a camera 8, serving as the endmost link. When the observation manipulator 3 is operated, the tip of the camera 8 can be oriented in any three-dimensional direction, thus allowing the imaging region of an operative site of the patient A to be changed freely.

As shown in FIG. 4, one manipulator 4 is a stabilizer manipulator (hereinafter also referred to as a stabilizer manipulator 4), equipped with a stabilizer 9, serving as the endmost link. When the stabilizer manipulator 4 is operated, the stabilizer 9 can be oriented in any three-dimensional direction, thus allowing the position at which the operative site to be held of the patient A is changed freely.

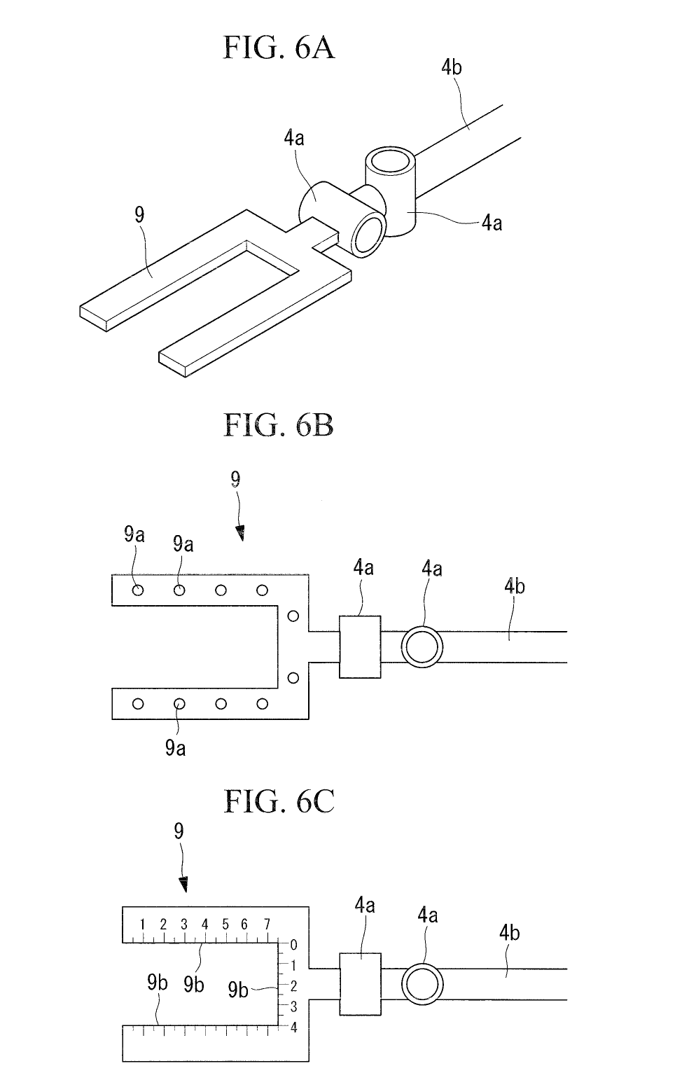

For example, as shown in FIG. 6A, the stabilizer 9 is formed of a U-shaped plate member. As shown in FIG. 6B, one side of the stabilizer 9 has suction holes 9a that are sucked under negative pressure to be attached to biological tissue around the operative site. As shown in FIG. 6C, the other side of the stabilizer 9 has a scale 9b drawn at intervals along the inner edge of the U-shape. The scale 9b functions as an indicator that makes it easy to specify a treatment site set inside the stabilizer 9.

For a minimally invasive surgery etc., it is preferable that the stabilizer 9 be formed in a foldable structure (not shown) so as to be inserted into the body through a trocar (not shown) disposed through the skin, and when passing through the trocar, the stabilizer 9 be folded small, and after it is inserted into the body, it be extended into the shape shown in FIGS. 6A to 6C.

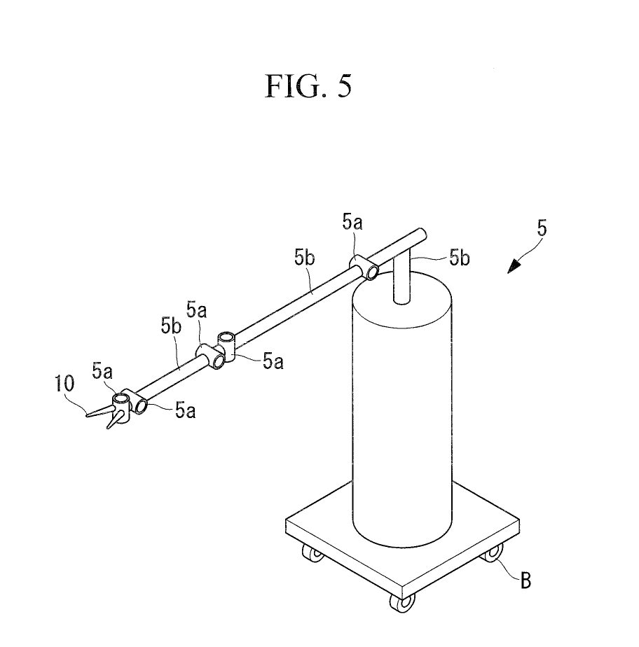

The other two manipulators 5 are, as shown in FIG. 5, operating manipulators (hereinafter, also referred to as operating manipulators 5) each equipped with an instrument 10, such as forceps, serving as the endmost link. When the operating manipulators 5 are operated, the instruments 10 can be disposed in any three-dimensional orientation, thus allowing access to the operative site of the patient A from a desired direction for treatment.

The four manipulators 3 to 5 are associated with one another using a common coordinate system.

The operating unit 7 is equipped with an input unit 7a, such as a joystick, that an operator X manipulates and a display 7b that displays an image acquired by the camera 8. One input unit 7a is provided for each of the manipulators 3 to 5 or is provided so as to manipulate all the manipulators 3 to 5 by switching between them using a switch (not shown).

The control unit 6 is equipped with a calculating section 6a that calculates the rotation angles of the joints 3a to 5a according to the input from the input unit 7a, driving sections 6b that calculate and output instruction signals to the driving units 3c to 5c on the basis of the rotation angles calculated by the calculating section 6a, an image processing section 6c that processes the image data acquired by the camera 8 to generate image information to be displayed on the display unit 7b, and a storage section 6d that stores the sizes of the links 3b to 5b of the respective manipulators 3 to 5.

The operator X controls the operating manipulators 5 by manipulating the input unit 7a of the operating unit 7 so as to operate the stabilizer manipulator 4 to hold the operative site with the stabilizer 9 and, in this state, to move the tip positions of the instruments 10 of the two operating manipulators 5 toward the vicinity of the target site P set to the stabilizer 9. Specifically, the operating angles of the joints 3a to 5a are calculated, and the driving units 3c to 5c are operated so that the operating angles calculated while detection signals from the sensors 3d to 5d are monitored can be achieved so that the tip positions of the instruments 10 can be disposed in the vicinity of the target site P set to the stabilizer 9 that holds the operative site.

The operation of the thus-configured surgical manipulator system 1 according to this embodiment will be described below.

To treat the operative site of the patient A using the surgical manipulator system 1 according to this embodiment, first, the operator X manipulates the input unit 7a to operate the observation manipulator 3 to adjust the orientation and position of the camera 8 so that the operative site falls within the imaging region of the camera 8.

Next, the operator X manipulates the input unit 7a to operate the stabilizer manipulator 4 to move the stabilizer 9 serving as the endmost link into the imaging region of the camera 8. Since the stabilizer 9 is provided with the scale 9b, the operator X can operate the input unit 7a with being moved the stabilizer manipulator while comparing the scale 9b displayed on the display unit 7b with the operative site so that the target site P set to the stabilizer 9 matches the treatment site of the operative site.

While the example shown in the drawing is described using one point as the target site P, a specified region P′ may be set to the stabilizer 9 as a target site, as shown in the drawing.

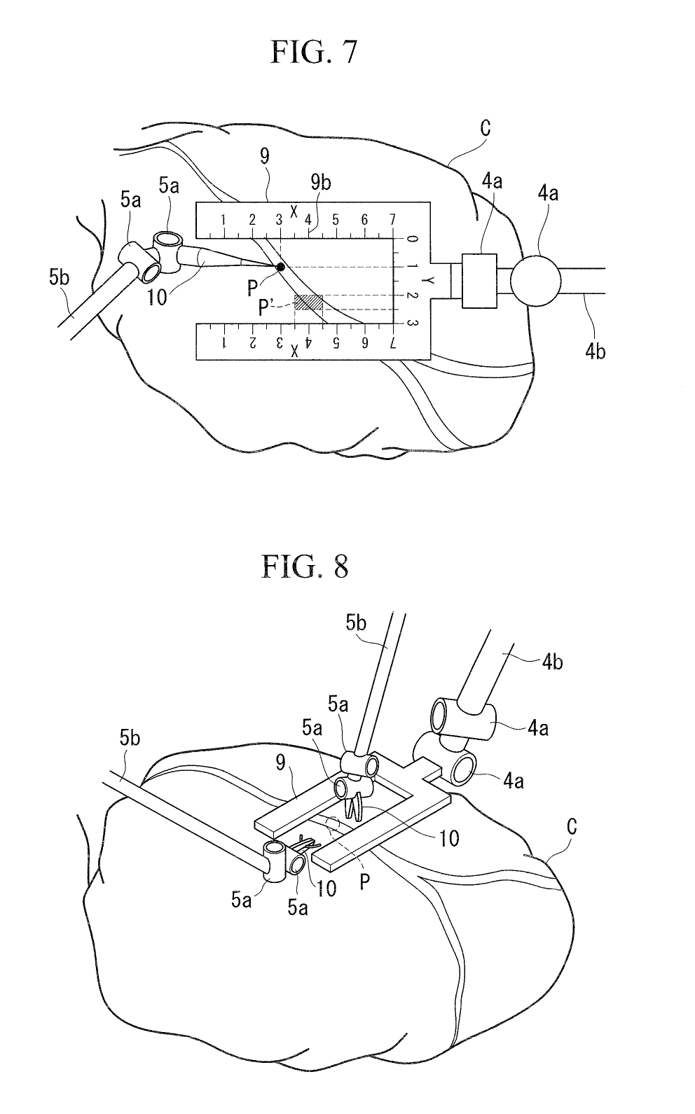

That is, for example, if coordinates (X, Y, Z)=(3, 1, 2) are set as the target site P using the scale 9b of the stabilizer 9, as shown in FIG. 7, the stabilizer 9 is moved so that the treatment site of an organ C exactly matches the position of the coordinates (X, Y, Z)=(3, 1, 2) of the scale 9b of the stabilizer 9, and at the position, the stabilizer 9 is fixed to the organ C. Here, the coordinates may be (X, Y)=(3, 1), using a predetermined value for the Z-axis direction. The Z-axis is defined as an axis orthogonal to the X-axis and the Y-axis.

The stabilizer 9 is attached around the treatment site of the organ C by sucking the suction holes 9a under negative pressure, with the stabilizer 9 in contact with the organ C so that no load is imposed thereon. Thus, even for an organ C such as the heart, the pulsation, such as the heartbeat, of the organ C, can be inhibited using the stabilizer 9.

Furthermore, with the surgical manipulator system 1 according to this embodiment, the control unit 6 controls the operating manipulators 5 so that the tips of the instruments 10 are disposed in the vicinity of the target site P that is aligned with the treatment site of the organ C using the stabilizer 9. That is, in the surgical manipulator system 1 according to this embodiment, because the coordinate systems of the manipulators 3 to 5 are associated with one another, and the sizes etc. of the links 3b to 5b that constitute the manipulators 3 to 5 are known, when the target site P is set, the operating angles of the joints 5a of the operating manipulators 5 can be calculated backward so that the tips of the instruments 10 are disposed in the vicinity of the target site P. Accordingly, after the stabilizer 9 is fixed at the desired position, the instruments 10 can be disposed in the vicinity of the treatment site of the organ C with a single motion, as shown in FIG. 8.

As a result, in a case where there are a plurality of the target sites P to be treated or in a case where the same target site P must be treated a plurality of times, for example, for an anastomosis treatment, the treatment can be performed automatically without the operator visually moving the instruments 10 as the need arises. Accordingly, this has the advantage that the operating manipulators 5 can be operated easily and quickly, thus reducing the burden imposed on the operator X.

In this embodiment, the indicator to specify the treatment site is formed of the scale 9b drawn on the surface of the stabilizer 9. As an alternative, it may be any kind of scale provided that it is formed on the surface of the stabilizer 9 (the surface that can be imaged) that comes into contact with the operative site so as to allow image recognition, and that it can show the coordinates of the operative site. As a shaped portion that substitutes for the scale, a characteristic portion of the shape of the stabilizer 9 main body (for example, a region including the tip and/or the near end of the stabilizer 9 that comes into contact with the operative site) may be set as an indicator region; and an indicator display unit (not shown) may be provided which calculates the target site P from the shape of the stabilizer 9 and which superimposes the calculated target site P itself or an image that shows an indicator for indirectly indicating the target site P on the image of the stabilizer 9 by performing image processing on the indicator region among images of the stabilizer 9 taken by the camera 8.

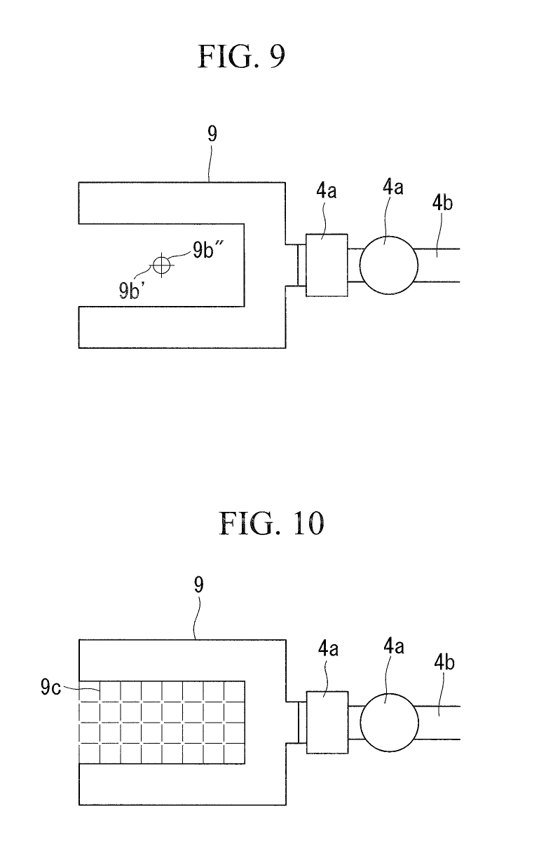

In this case, a characteristic design (protrusion, notch, etc.) that makes it easy to recognize an image of the indicator region may be provided around the outer edge of the stabilizer 9. As an indicator that indicates the target site P itself, a cross-shaped indicator 9b′ or a circular indicator 9b″, as shown in FIG. 9, may be used. As an indicator for indirectly indicating the target site P, the scale 9b or ruled lines 9c may be provided, which is advantageous in that they are superimposed on the inner edge of the stabilizer 9, as shown in FIGS. 6C and 10, respectively; however, it is preferable to provide a scale that indicates a coordinate system having the same scale as coordinates set for the image of the stabilizer 9. In addition to the above-described various indicators, a registration indicator that indicates a reference position for matching an indicator image with the image of the stabilizer 9 may be drawn on the surface of the stabilizer 9.

In this embodiment, the present invention is applied to the surgical manipulator system 1 having two operating manipulators 5. However, instead of this, the present invention may be applied to a surgical manipulator system 1 having one or three or more operating manipulators 5.

What is claimed is:

1. A surgical manipulator system comprising:
a stabilizer manipulator having a stabilizer, at the tip, holding an operative site of a patient;
one or more operating manipulator having an instrument, at the tip, treating the operative site held by the stabilizer manipulator; and
a control unit controlling these manipulators, wherein
the stabilizer manipulator and the operating manipulator have coordinate systems that are associated with one another; and
the position coordinates of a target site to be treated are set to the stabilizer provided on the stabilizer manipulator; and
the control unit controls the operating manipulator so as to dispose the instrument in the vicinity of the target site set to the stabilizer when the stabilizer is disposed at the operative site to hold the operative site.

2. The surgical manipulator system according to claim 1, further comprising:
an observation manipulator having a camera, at the tip, for observing the operative site and having a coordinate system that is mutually associated with the stabilizer manipulator;
a display unit displaying an image of the operative site acquired by the camera of the observation manipulator; and
an indicator display unit displaying an indicator for specifying the target site in the image displayed on the display unit.

3. The surgical manipulator system according to claim 2, wherein the indicator is a shaped portion provided on the surface of the stabilizer.

4. The surgical manipulator system according to claim 3, wherein the shaped portion is a scale provided on the surface of the stabilizer.

5. The surgical manipulator system according to claim 3, wherein the shaped portion is a characteristic shape formed on the main body of the stabilizer.

6. The surgical manipulator system according to claim 2, wherein the indicator display unit superimposes an image of the indicator set to the stabilizer on the image of the operative site.

7. The surgical manipulator system according to claim 6, wherein the image of the indicator is a scale.
